(19) United States
(12) Patent Application Publication
(10) Pub. No.: US 2026/0248542 A1
(43) Pub. Date: Aug. 27, 2026

(54) Percutaneous Inserter System For Bone Fixation Members, And Related Instrumentation, Assemblies, And Methods

(71) Applicant: DePuy Synthes Products, Inc., Raynham, MA (US)

(72) Inventors: Joel Oberli, Niederdorf (CH); This Aebi, Grenchen (CH)

(21) Appl. No.: 19/062,196

(22) Filed: Feb. 25, 2025

(51) Int. Cl. A61B 17/80

(52) U.S. Cl. CPC A61B 17/808

(57) ABSTRACT

A surgical system includes an outer tube elongate along a longitudinal direction and an inner tube having an insertion portion configured to insert within a cannulation of the outer tube. The cannulation extends between proximal and distal ends of the outer tube along a central longitudinal axis. The outer tube has a flexible portion that extends to the distal end and defines at least one spring member configured to flex in a radial direction perpendicular to the central axis between: (1) a neutral configuration, in which the distal end defines a first radial dimension, and (2) a expanded configuration, in which the distal end defines a second radial dimension greater than the first radial dimension. The insertion portion of the inner tube body is configured to contact the flexible portion of the outer tube body in a manner that flexes it into the expanded configuration.

### TECHNICAL FIELD

The present invention relates to bone fixation, particularly a percutaneous inserter system for guiding bone fixation members to a target location through an associated fixation hole in a bone plate, and related instrumentation, assemblies, and methods.

### BACKGROUND

Bone plate systems for the internal fixation of bone fractures are well known. Conventional bone plate systems are particularly well-suited to promote the healing of a fracture. A bone fixation member, such as a bone screw, is inserted through a fixation aperture or hole in a bone plate and is threaded into bone to compress, neutralize, buttress, tension, band, and/or bridge the fracture ends together. Cannulas can be employed with the bone plating system to facilitate insertion of the bone fixation member into the bone while reducing contact with the soft tissue through which the bone fixation member must pass.

### SUMMARY

According to an embodiment of the present disclosure, a surgical system includes an outer tube elongate along a longitudinal direction and an inner tube having an insertion portion configured to insert within a cannulation of the outer tube. The cannulation extends between proximal and distal ends of the outer tube along a central longitudinal axis. The outer tube has a flexible portion that extends to the distal end and defines at least one spring member configured to flex in a radial direction perpendicular to the central axis between: (1) a neutral configuration, in which the distal end defines a first radial dimension, and (2) a expanded configuration, in which the distal end defines a second radial dimension greater than the first radial dimension. The insertion portion of the inner tube body is configured to contact the flexible portion of the outer tube body in a manner that flexes it into the expanded configuration.

According to another embodiment of the present disclosure, a method for preparing a sleeve assembly to provide passage for a fixation member includes inserting an inner tube body within a cannulation of an outer tube body along a central axis of the outer tube body. During the inserting step, an insertion portion of the inner tube body engages an expandable distal portion of the outer tube body; and a distal end of the inner tube body extends distally from a distal end of the outer tube body at an offset distance. The method includes expanding the expandable distal portion of the outer tube body along a radial direction perpendicular to the central axis via the engagement with the insertion portion of the inner tube body. After the expanding step, a fixation member is translatable along the central axis and through the cannulation. The method also includes orienting the outer tube body, wherein the central axis intersects a fixation hole of a plate after the inserting step.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a perspective view of a surgical system that includes an outer tube body and an inner tube body coupled together for guiding instrumentation and/or a bone fixation member through soft tissue relative to a bone plate, according to an embodiment of the present disclosure;

FIG. 1B is a plan view showing additional components of the surgical system for use with the outer tube body illustrated in FIG. 1A, according to an embodiment of the present disclosure;

FIG. 2A is an exploded perspective view of a bone plate and a bone fixation member relative to an underlying bone;

FIG. 2B is a side view of the bone fixation member illustrated in FIG. 2A;

FIG. 2C is an enlarged sectional side view of a head of the bone fixation member illustrated in FIG. 2B;

FIG. 2D is a perspective view of a fixation hole of the bone plate illustrated in FIG. 2A;

FIGS. 2E and 2F are sectional side views of the bone fixation member affixed within the fixation hole illustrated in FIG. 2D at a nominal orientation (FIG. 2E) and at an angulated orientation (FIG. 2F);

FIG. 3A is a perspective view of the outer tube body illustrated in FIG. 1A, according to an embodiment of the present disclosure;

FIG. 3B is a sectional perspective view of the outer tube body illustrated in FIG. 3A;

FIG. 3C is an enlarged sectional side of a flexible distal region of the outer tube body illustrated in FIG. 3A in a neutral configuration;

FIG. 3D is an enlarged sectional side of a flexible distal region of the outer tube body in an expanded configuration;

FIG. 4A is a perspective view of an inner tube body for assembly with the outer tube body illustrated in FIG. 3A, according to an embodiment of the present disclosure;

FIG. 4B is a perspective view of the inner tube body illustrated in FIG. 4A assembled with the outer tube body illustrated in FIG. 3A;

FIG. 4C is a sectional side view of the assembled inner and outer tube bodies illustrated in FIG. 4B, having a trocar inserted through the inner tube body, according to an embodiment of the present disclosure;

FIG. 4D is a sectional side view of the assembled inner and outer tube bodies illustrated in FIG. 4B, having a drill bit inserted through the inner tube body, according to an embodiment of the present disclosure;

FIGS. 5A and 5B are sectional side views of a locking mechanism of the assembled inner and outer tube bodies illustrated in FIG. 4B, shown in a locked configuration (FIG. 5A) and an unlocked configuration (FIG. 5B), according to an embodiment of the present disclosure;

FIG. 5C is a sectional side view of a distal region of the assembled inner and outer tube bodies illustrated in FIG. 4B, with the flexible region of the outer tube body in an expanded configuration and the distal region of the inner tube body seated within a fixation hole, according to an embodiment of the present disclosure;

FIG. 5D is a sectional side view of the flexible distal region of the outer tube body illustrated in FIG. 4B, shown in a neutral configuration and seated within the fixation hole, according to an embodiment of the present disclosure;

FIG. 5E is a sectional side view of a secondary locking mechanism (shown in a locked configuration) for locking a trocar to the inner tube body illustrated in FIG. 4A, according to an embodiment of the present disclosure;

FIGS. 6A-6E show a series of steps during use of the outer tube body illustrated in FIG. 3A to insert a bone fixation member through a fixation hole of a bone plate and into underlying bone, according to an embodiment of the present disclosure;

FIG. 7A is a perspective view of an another inner tube body for assembly with the outer tube body illustrated in FIG. 3A, according to an embodiment of the present disclosure;

FIG. 7B is a perspective view of the inner tube body illustrated in FIG. 4A assembled with the outer tube body illustrated in FIG. 3A;

FIG. 7C is a sectional side view of a proximal region of the assembled inner and outer tube bodies illustrated in FIG. 7B;

FIG. 7D is a sectional side view of a distal region of the assembled inner and outer tube bodies illustrated in FIG. 7B, shown with the distal region of the inner tube body against a bone plate and above a bone fixation member affixed to the bone plate, according to an embodiment of the present disclosure;

FIGS. 8A-8D show a series of steps during use of the assembled inner and outer tube bodies illustrated in FIG. 7B to remove a bone fixation member from a bone plate and from underlying bone, according to an embodiment of the present disclosure;

FIG. 9 is a perspective view showing an outer tube body having an alternative, knob-like handle, according to an embodiment of the present disclosure;

FIGS. 10A and 10B are perspective views showing an outer tube body having another alternative handle, which includes a pair of rotatable arms for supporting the outer tube body relative to patient anatomy, according to an embodiment of the present disclosure;

FIG. 11 is a diagram plan view showing an exemplary method of using an outer tube body and an inner tube body, according to an embodiment of the present disclosure;

FIG. 12A is a diagram plan view showing another exemplary method of using an outer tube body and an inner tube body, according to an embodiment of the present disclosure;

FIG. 12B is a diagram plan view showing an exemplary use of the method shown in FIG. 12A for inserting a bone fixation member into patient anatomy; and

FIG. 12C is a diagram plan view showing an exemplary use of the method shown in FIG. 12A for removing a bone fixation member from patient anatomy.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, assemblies, systems, methods, applications, conditions or parameters described and/or shown herein, and that the terminology used herein is for the purpose of describing particular embodiments by way of example only and is not intended to be limiting of the scope of the present disclosure.

The embodiments disclosed herein pertain to surgical systems involving assemblies of tubular bodies that are employed to facilitate one or more of (1) insertion of a bone fixation member through soft tissue to a target location within bone, and (2) removal through soft tissue of a previously inserted bone fixation member within bone. The embodiments herein include an outer tubular body having a flexible distal region for use with various inner sleeves. The inner sleeves are configured to expand the flexible distal region of the outer tubular body radially outward to allow passage therethrough for bone fixation members to and/or from the treatments site, and also passage for surgical instrumentation (e.g., trocars, drill bits, measuring devices (e.g., calibrated drill bits), drivers) for interacting with the treatment site and/or for interacting with the bone fixation member. The radial expandability of the flexible distal region of the outer tubular body allows for passage of wider inner sleeves and instruments and for better access to the treatment site. Additionally, the inner sleeves are configured to provide enhanced feedback (e.g., tactile feedback) pertaining to the positioning of the tubular bodies relative to patient anatomy and/or implanted structures (e.g., bone plates) within the patient anatomy. Moreover, the outer and inner tubes are configured to provide seamless interchanging of the inner sleeves as needed during surgery.

As used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although terms involving numerical prepositions (e.g., “first,” “second,” “third”) can be used herein to describe various features, such features should not be limited by these terms. These terms are instead used to distinguish one feature from another. For example, a first element could be termed a second element in another context, and, similarly, a second element could be termed a first element in another context, without departing from the scope of the embodiments disclosed herein.

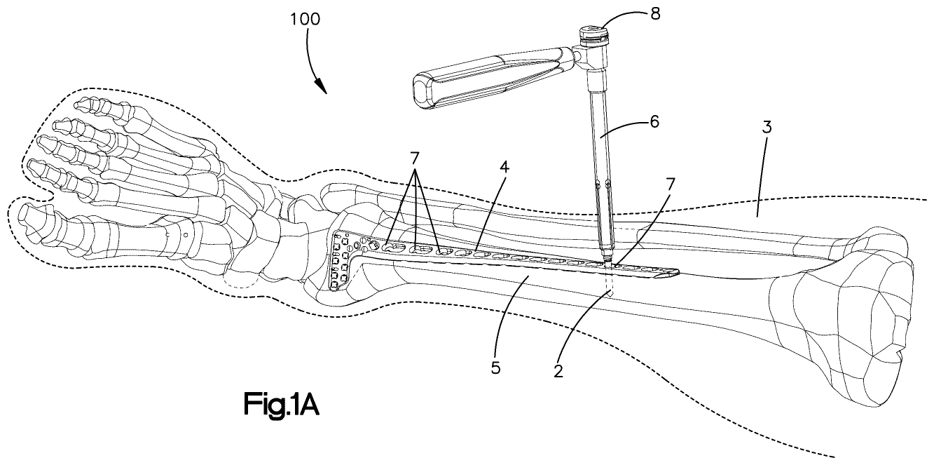

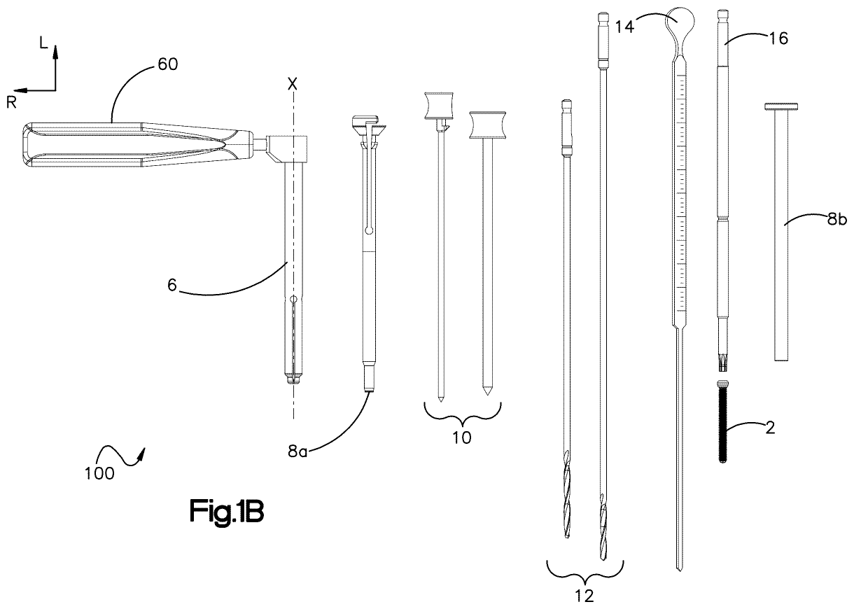

Referring now to FIGS. 1A-1B, an exemplary surgical system 100 is shown for guiding bone fixation members 2 through soft tissue 3 relative to a bone plate 4 that interfaces with underlying bone 5 at a surgical treatment site. The surgical system 100 includes an outer protection sleeve 6 and one or more inner sleeves 8 configured for insertion within, and coupling with, the outer protection sleeve 6. In particular, the coupled outer and inner sleeves 6, 8 are adapted for targeting one or more select features of the bone plate 4, such as a fixation hole 7. As used herein, the outer protection sleeve 6 can also be referred to as an “outer tube body” 6 (or simply an “outer tube” 6) and the one or more inner sleeves 8 can each also referred to an “inner tube body” 8. The surgical system 100 includes a bone plate 4, one or more bone fixation members 2 (such as bone screws 2), and instrumentation for insertion through the outer tube 6 to interact with the treatment site. As shown in FIG. 1B, such instrumentation can include hole opening devices, such as one or more trocars 10 and one or more drill bits 12, for creating and/or expanding a hole in the underlying bone 5 at the treatment site. The instrumentation can also include one or more measuring instruments, such as depth-measuring instruments, such as calibrated drill bits 14, for determining the desired size of bone fixation member 2 for use. The instrumentation can also include one or more drivers 16 for coupling with and moving the bone fixation member(s) 2 through the outer tube 6 relative to the underlying bone 5. It should be appreciated that the surgical system 100 can include various additional instruments for interacting with the treatment site, such as Kirschner wires (K-wires), guide wires, pins, and radiopaque markers, by way of non-limiting examples.

The outer tube 6 is elongate along a longitudinal direction L and defines a central axis X oriented along the longitudinal direction L. A radial direction R intersects the central axis X and is perpendicular thereto. The inner tube bodies 8 define respective central axes and are configured for coaxial insertion within the outer tube 6. Additionally, the instruments 10, 12, 14, 16 described above are also configured for substantially coaxial insertion through a respective inner tube body 8 and the outer tube 6. Thus, although the central axis X and the longitudinal and radial directions L, R are defined by the outer tube 6, they can be respectively characterized as the central axis and longitudinal and radial directions of the outer tube bodies 8 and instruments of the surgical system 100 during use. Additionally, it should be appreciated that, as used herein: the terms “longitudinal”, “longitudinally”, and derivatives thereof refer to the longitudinal direction L; and the terms “radial”, “radially”, and derivatives thereof refer to the radial direction R.

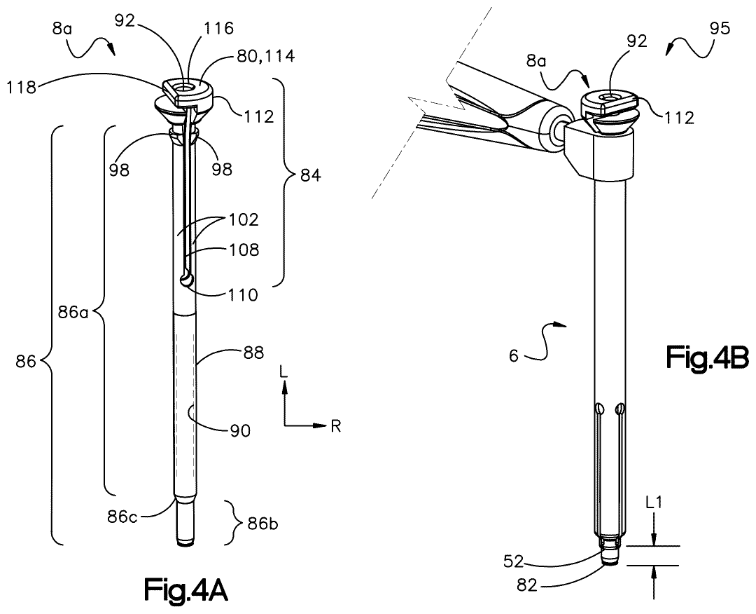

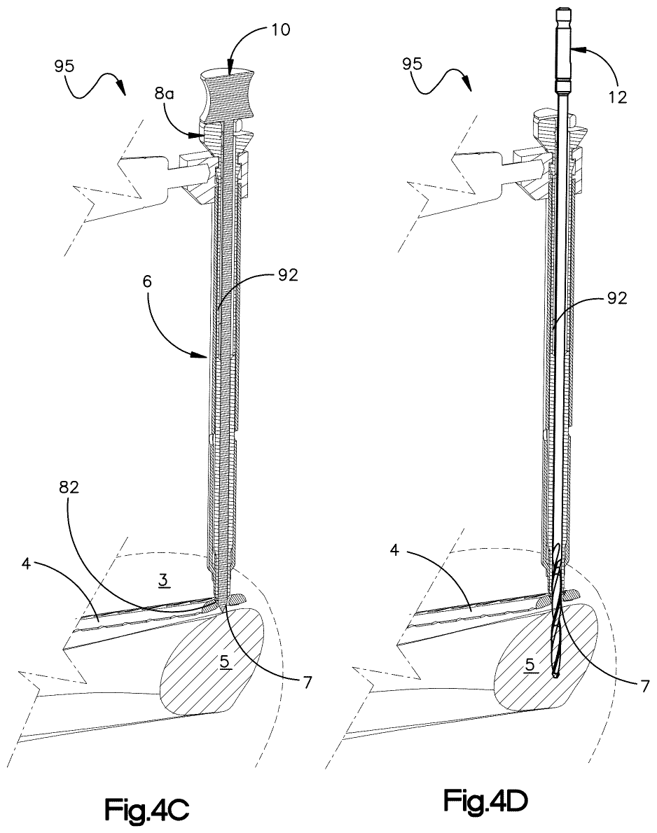

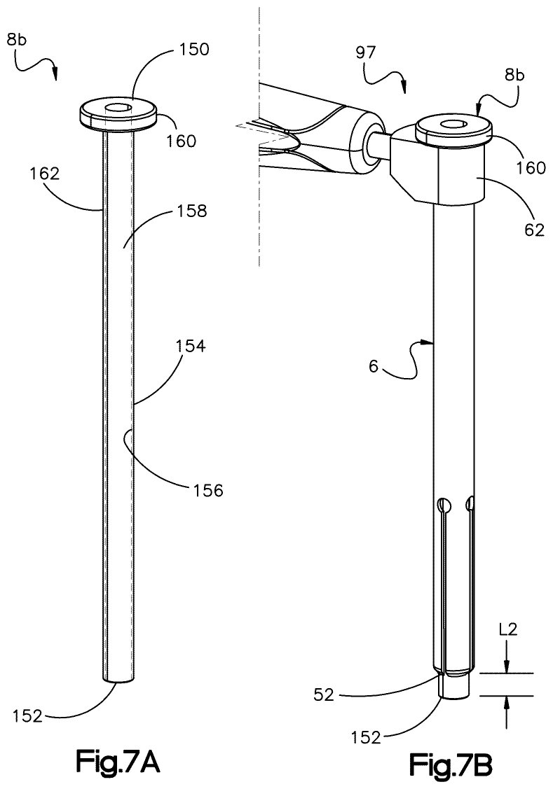

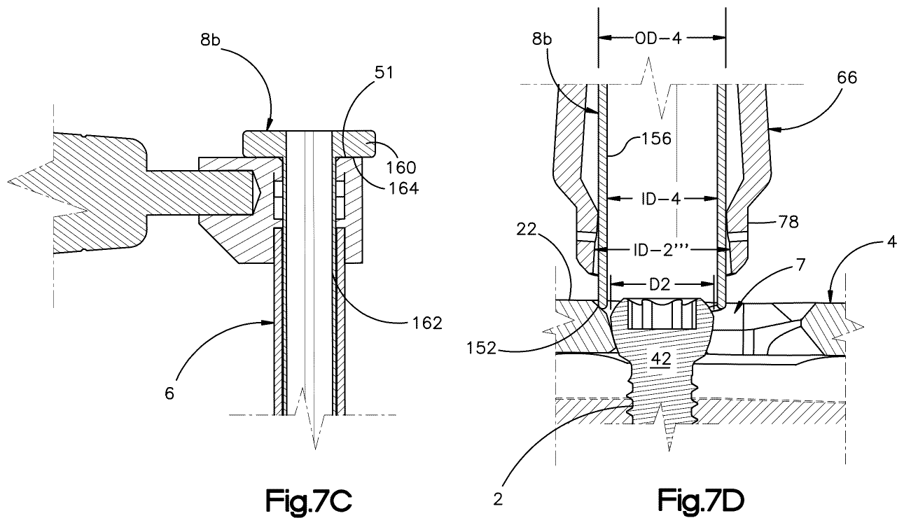

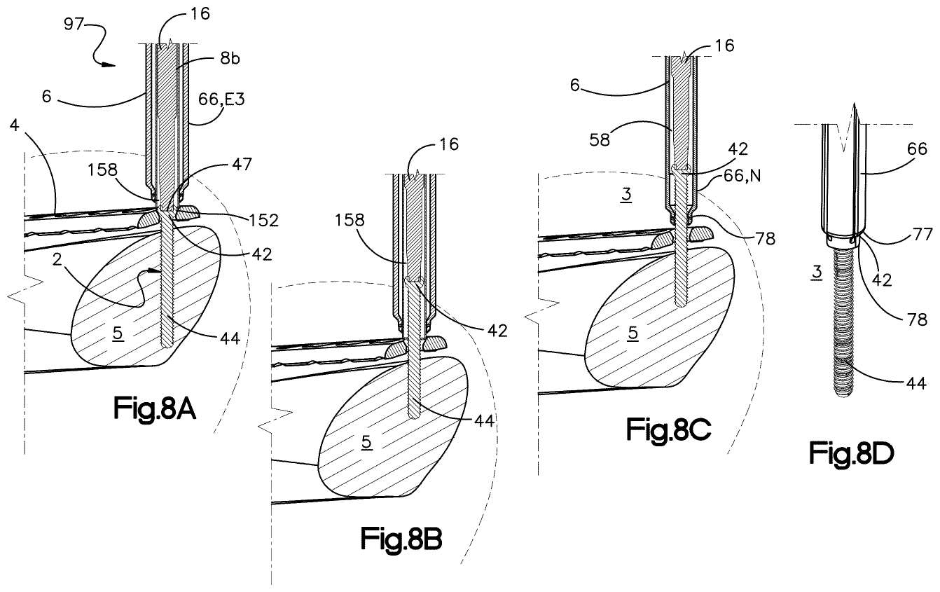

In the illustrated embodiments herein, the inner tube bodies 8 include two (2) types of inner sleeves: (1) inner guide sleeves 8a, such as guide sleeves 8a configured for guiding instrumentation toward the underlying bone 5, and (2) inner removal sleeves 8b configured for facilitating removal of a bone fixation member 2 from the underlying bone 5. The outer tube 6 and the inner tube bodies 8a, b are preferably cooperatively configured such that the inner tube bodies 8a, b are interchangeably attachable to the outer tube body 8 as needed, as discussed in more detail below. Aspects of the surgical system 100 relating to use of the outer tube 6 with an inner guide sleeve 8a will be described with reference to FIGS. 4A-6E, and aspects relating to use of the outer tube 6 with a removal sleeve 8b will be described with reference to FIGS. 7A-8D.

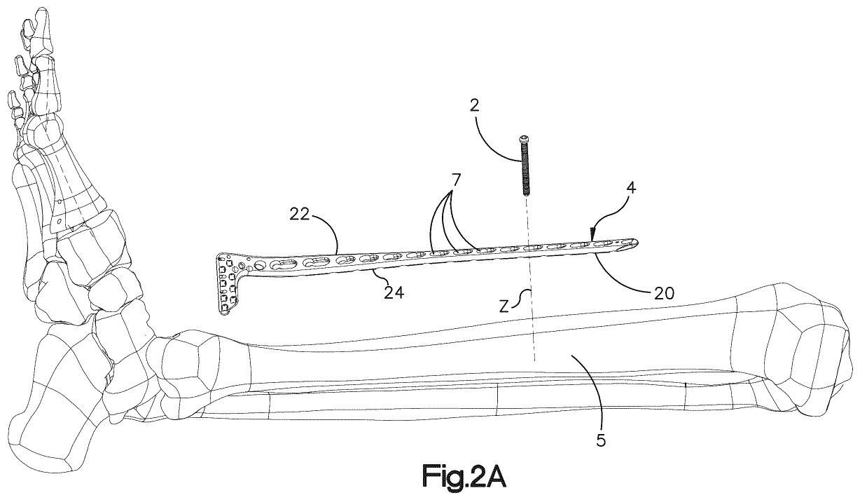

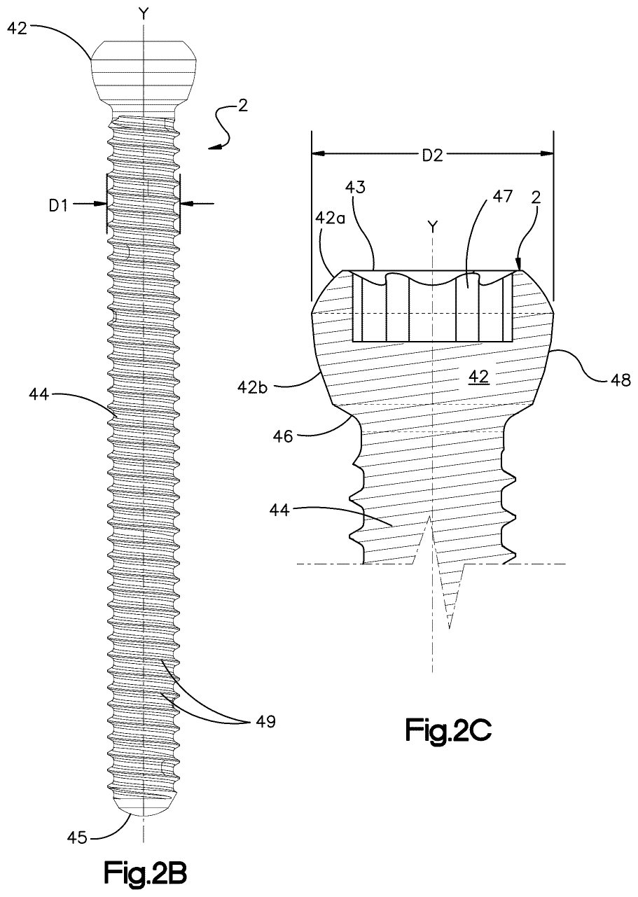

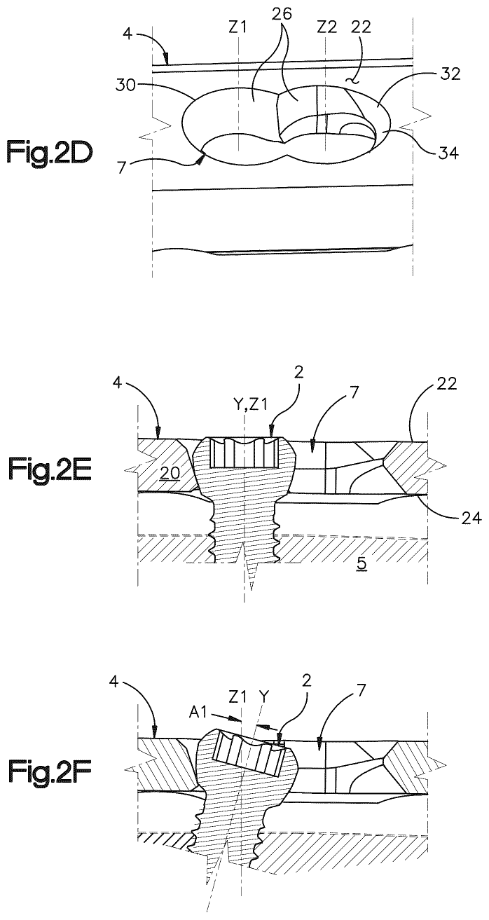

Referring now to FIGS. 2A-2C, an exemplary bone plate 4 and bone fixation member 2 of the surgical system 100 are shown for fixation with underlying bone 5. The bone plate 4 has a plate body 20 that defines an outer plate surface 22 configured to face away from the underlying bone 5 and a bone-facing surface 24 configured to face the underlying bone 5. The plate body 20 defines one or more fixation holes 7 that extend from the outer plate surface 22 to the bone-facing surface 24 along one or more respective central hole axes Z. The fixation holes 7 are configured for receiving respective bone fixation members 2 for affixing the bone plate 4 to the underlying bone 5.

It should also be appreciated that the term “underlying”, particularly as used herein with reference to bone that interfaces with a bone plate 4, means positioned distally of the bone plate, i.e., the underlying bone 5 faces the bone-facing surface 24 and is remote from the outer surface 22 of the bone plate 4. Furthermore, although the illustrated embodiments herein depict the bone plate 4 positioned generally vertically above the underlying bone 5, the positions of the bone plate 4 and underlying bone 5 in three-dimensional (3D) space depend upon the position and orientation of the associated patient anatomy in 3D space. For example, the patient can be positioned such that the bone plate 4 and underlying bone 5 are horizontally spaced from each other, or such that the underlying bone 5 is positioned above the bone plate 4 in 3D space.

As shown in FIG. 2B-2C, the bone fixation members 2 of the illustrated embodiment are bone screws 2 each having a screw body 40 that defines a head 42 and a shaft 44 extending from the head 42 along a central screw axis Y to a distal screw tip 45. The screw head 42 extends longitudinally along the central screw axis Y from a proximal end 43 of the head 42 to a neck 46 that effectively defines a distal end of the head 42 and shares a boundary with the shaft 44. The head 42 defines a drive socket 47 that extends distally from the proximal head end 43 and is configured to receive a distal end of a driver 16. The head 42 defines an outer head surface 48 that extends between the proximal head end 43 and the neck 46. The outer head surface 48 is configured to engage the plate body 20 within a respective fixation hole 7. Although the outer head surface 48 of the illustrated embodiment is generally smooth, it should be appreciated that the outer head surface 48 can define external threading, such as for threadedly engaging interior threads of a fixation hole 7 for locking therewith. The screw shaft 44 defines external threads 49 configured to engage bone material for affixing the bone screw 2 to the bone. In particular, the screw shaft 44 is configured to extend through a fixation hole 7 and engage underlying bone material until the head 42 is fully seated within the fixation hole 7. It should be appreciated that in addition to or as an alternative to the external threads 49, the screw shaft 44 can define other features for engaging bone material, such as cutting teeth, flutes, and the like.

The outer tube 6 is configured to have features that are complementary with the geometries of the head 42 and shaft 44 of the bone screw 2. As shown in FIG. 2B, the screw shaft 44 has a maximum shaft outer dimension D1, which in the illustrated embodiment is defined by the major diameter D1 of the external threads 49. As shown in FIG. 2C, the head 42 has a proximal head portion 42a and a distal head portion 42b that are longitudinally spaced from each other along the central screw axis Y. The proximal head portion 42a extends proximally to the proximal head end 43. The distal head portion 42b extends distally away from the proximal head portion 42a to the neck 47. The outer head surface 48 defines a maximum head outer dimension D2, which is preferably located at an interfacing boundary between the proximal and distal head portions 42a, b. The outer head surface 48 of the proximal head portion 42a preferably tapers radially inwardly and proximally, and the distal head portion 42b preferably tapers radially inwardly and distally, which facilitates, among other things, advantageous complementary engagement with the outer tube 6, as described in more detail below.

The bone screws 2 can include locking-type bone screws 2 configured for locking engagement with the fixation hole 7, such as variable-angle (VA) locking screws (i.e., screws configured to lock within a fixation hole at either a nominal orientation or an “angulated” orientation whereby the central screw axis Y is oriented at an acute angle with respect to the respective central hole axis Z) or standard-type locking screws (i.e., screws configured to lock with the fixation hole at a nominal orientation whereby the central screw axis is substantially aligned with the central hole axis). The bone screws 2 can also include compression-type bone screws for contacting a compression surface within the fixation hole to drive dynamic compression of the bone plate 4. Compression-type bone screws can also be employed for insertion within a fixation hole 7 (e.g., a locking hole, compression hole, or combination hole) described herein to compress the bone plate 4 to the underlying bone without causing dynamic compression). Although the bone screws 2 of the illustrated embodiments are configured for insertion within a pre-drilled or otherwise pre-formed hole in the underlying bone, it should be appreciated that the embodiments herein can be adapted for use with self-drilling bone screws. It should also be appreciated that the embodiments herein can be adapted for use with other types of bone fixation members, such as spiral blades, pins, nails, and wires (e.g., guide wires for use with cannulated bone screws), by way of non-limiting examples.

As shown in FIGS. 2D-2E, the fixation holes 7 are configured such that the shaft 44 advances through the hole 7 and into the underlying bone 5 until the head 42 seats against the plate body 20 within the hole 7. Within each fixation hole 7, the plate body 20 defines an interior hole surface 26 that is configured to engage the head 42 of a respective screw 2. The interior hole surfaces 26 of the fixation holes 7 include upper hole portions adjacent the outer plate surface 22, which upper hole portions taper inwardly toward the central hole axis Z and toward the bone-facing surface 24 and are configured to provide a seat for the screw head 42. The interior hole surface 26 can define locking structures, such as threading, columns, and/or recesses, for locking with interfacing features of the screw head 42, such as exterior threading. As shown, one or more of the fixation holes 7 can be a combination hole (also referred to as a “combi-hole”) having a first, locking hole portion 30 that includes locking structure(s) and a second, compression hole portion 32 in which the interior hole surface 26 includes a compression surface 34. In such combi-holes, the locking hole portion 30 extends through the plate body 20 along a locking central hole axis Z1, and the compression hole portion 32 extends through the plate body 20 along a central compression hole axis Z2. It should be appreciated that the bone plate 4 can also include one or more fixation holes 7 that are designated locking holes and one or more fixation holes 7 that are designated compression holes.

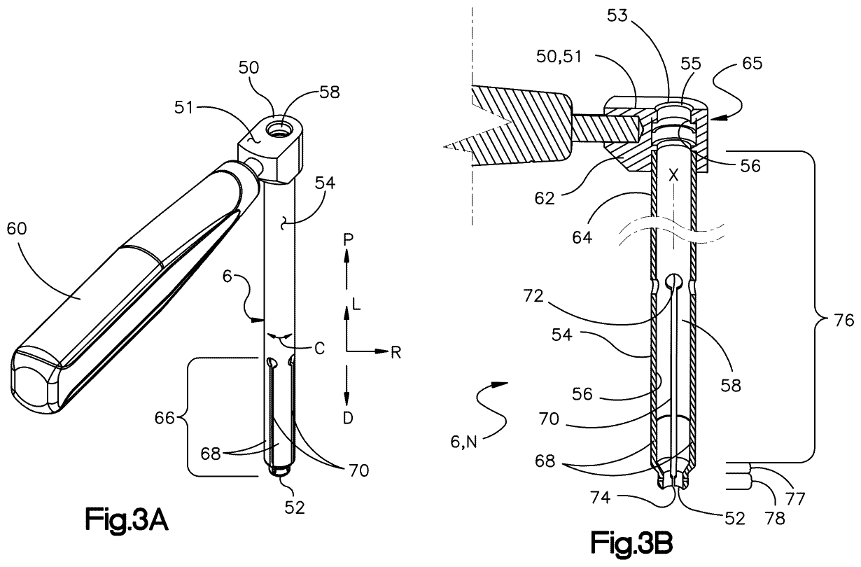

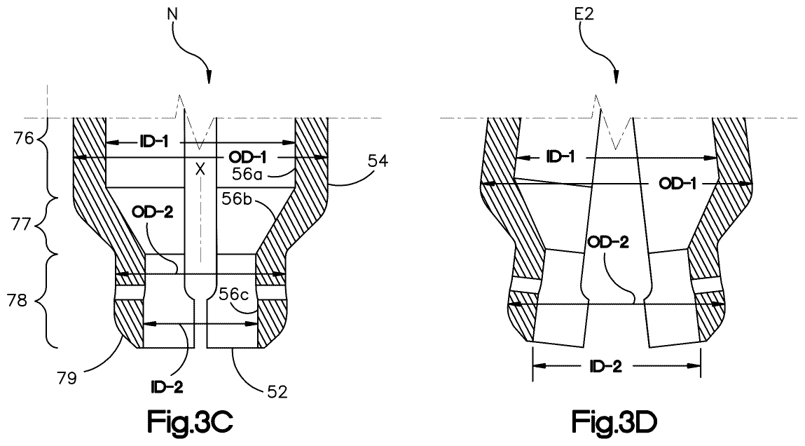

Referring now to FIGS. 3A-3B, the outer tube 6 has a proximal end 50 and a distal end 52 spaced from the proximal end 50 in a distal direction D oriented along the longitudinal direction L. The proximal end 50 is spaced from the distal end 52 in a proximal direction P opposite the distal direction D. It should be appreciated that the proximal and distal direction P, D are each mono-directional components of the longitudinal direction L, which is bi-directional. It should also be appreciated that, as used herein, the terms “proximal”, “proximally”, and derivatives thereof refer to the proximal direction P, and the terms “distal”, “distally”, and derivatives thereof refer to the distal direction D. The outer tube 6 also has an outer surface 54 and an inner surface 56 opposite each other in the radial direction R. The inner surface 56 defines a cannulation 58 of the outer tube 6 that extends longitudinally from the proximal end 50 to the distal end 52 along the central axis X. The cannulation 58 is configured to provide passage for one or more of the inner tube bodies 8a, b, a select bone screw 2, and select instruments 10, 12, 14, 16 through soft tissue toward an associated fixation hole 7 and the underlying bone 5. The outer tube 6 preferably includes, or is at least attachable to, a handle member 60 that facilitates manual manipulation of the outer tube 6. In the illustrated embodiment, the outer tube 6 comprises a plurality of members coupled or otherwise joined together. For example, the outer tube 6 can include a cannulated proximal member 62 and a tube member 64 coupled with and extending distally from the proximal member 62. In other embodiments, however, the outer tube 6 can be a monolithic structure.

The outer tube 6 includes a coupling structure 65 for coupling with one or more of the inner tube bodies 8a, b. The coupling structure 65 preferably includes a mounting surface 51 configured to interface with associated mounting surfaces of the inner tube bodies 8a, b. As shown, the mounting surface 51 of the outer tube 6 can be a substantially planar landing surface at the proximal end 50 and can be substantially orthogonal to the central axis X. The coupling structure 65 is discussed in more detail below. The mounting surface 51 can also define a proximal opening 53 of the cannulation 58. The coupling structure 65 can also define one or more tapered lead-in surfaces 55 that extend from the mounting surface 51 to the inner surface 56.

The distal end 52 of the outer tube 6 is configured to be radially expandable during use. For this purpose, the outer tube 6 has a flexible distal portion 66 that extends to the distal end 52. The radial expandability of the flexible distal portion 66 is provided by at least one spring member 68 defined by the outer tube 6. The at least one spring member 68 is configured to allow the flexible distal portion 66 to flex radially between a neutral configuration N (shown in FIGS. 3A-3B) and one or more expanded configurations E1, E2, such as a first expanded configuration E1 and a second expanded configuration E2, as described in more detail below. The at least one spring member 68 is also preferably configured to allow the flexible distal portion 66 to flex radially inward from the neutral configuration N.

The at least one spring member 68 can be at least partially defined by slots or cutouts 70 extending radially through the outer tube 6 from the outer surface 54 to the inner surface 56. For example, in the illustrated embodiment, the at least one spring member 68 includes a plurality of longitudinal spring relief slots 70 extending distally from respective proximal slot ends 72 to the distal end 52 of the outer tube 6. Thus, the distal end 52 of the outer tube 6 also effectively defines distal slot ends 74 of the longitudinal spring relief slots 70. The presence of the spring relief slots 70 provides the flexible distal portion 66 with a plurality of spring members or arms 68 that are spaced from each other in a circumferential direction C about the central axis X. The spring relief slots 70 provide the spring members 68 with flexibility that facilitates the radial expansion from the neutral configuration N to the one or more expanded configurations E1, E2. The flexibility of the spring members 68 also facilitates radial contraction from the neutral configuration N. Such radial contraction can occur until circumferentially adjacent spring members 68 contact one another (i.e., until the spring relief slots 70 become circumferentially closed at the distal end 52).

As shown, the plurality of spring relief slots 70 can include four (4) slots 70 evenly spaced from each other (i.e., at 90-degree intervals) about the central axis X, thereby providing four (4) spring members 68 spaced about the central axis X. In other embodiments, however, the longitudinal spring relief slots 70 can include two (2), three (3), five (5), or more than five (5) slots 70, some of which and up to all which can be unevenly spaced from each other about the central axis X.

Additionally, the distal end 52 of the outer tubular body 6 is preferably configured to seat within one or more of the fixation holes 7, respectively, during select phases of use. For example, as shown in FIG. 3B, the outer tube 6 can have a main tube portion 76 and a distal tube portion 78 that is distally spaced from the main tube portion 76 and has a reduced radial dimension relative to a main tube portion 76 when in the neutral configuration N. The main tube portion 76 extends longitudinally between the proximal and distal ends 50, 52, such that the distal tube portion 78 is distally spaced from the main tube portion 76 and extends to the distal end 52. The outer tube 6 can also define an intermediate, step-down tube portion 77 located longitudinally between the main tube portion 76 and the distal tube portion 78.

Referring now to FIG. 3C, when the outer tube 6 of the illustrated embodiment is in the neutral configuration N, the inner surface 56a of the main tube portion 76 and the inner surface 56c of the distal tube portion 78 extend substantially along the longitudinal direction L (i.e., substantially parallel with the central axis X). The inner surface 56b of the step-down tube portion 77 preferably tapers inwardly and distally, which facilitates centering the bone screw 2 along the central axis X during screw insertion through the cannulation 58, as described in more detail below.

The distal tube portion 78 can include a tapered distal tip surface 79 that tapers radially inwardly and distally and is configured to seat within the fixation hole 7. The tapered distal tip surface 79 preferably has a rounded profile in an axial reference plane (i.e., a reference plane coextensive with the central axis X1), which facilitates smooth angulation of the outer tube 6 while in contact with the interior hole surface 26. The surgical system 100 of the illustrated embodiment is configured to facilitate angulated pre-drilling and bone screw 2 fixation within the fixation hole 7 at an acute angle A1 (also referred to as the “angulation A1”). The angulation A1 can be within a range from substantially nominal (0-degrees) to about 15 degrees or greater than 15 degrees with respect to the central hole axis Z. It should be appreciated that the angulation range substantially defines a cone extending an entire revolution (360-degrees) about the central hole axis Z. It should also be appreciated that the surgical system 100 facilitates eccentric angulation through the fixation hole 7 (i.e., where the central axis X of the outer tube 6 extends through the hole 7 at an offset distance from the central hole axis Z.

With continued reference to FIG. 3C, various radial dimensions of the outer tube 6 will now be described with respect to the neutral configuration N. The main tube portion 76 defines a first neutral outer dimension OD-1 and a first neutral inner dimension ID-1, both measured along the radial direction R. The first neutral outer dimension OD-1 is measured between opposing portions of the outer surface 54 along the main tube portion 76. The first neutral inner dimension ID-1 is measured between opposing portions of the inner surface 56a of the main tube portion 76. The distal tube portion 78 defines a second neutral outer dimension OD-2 and a second neutral inner dimension ID-2 along the radial direction R. The second neutral outer dimension OD-2 is measured between opposing portions of the outer surface 54 of the distal tube portion 78 and is less than the first neutral outer dimension OD-1. The second neutral inner dimension ID-2 is measured between opposing portions of the inner surface 56c of the distal tube portion 78 and is preferably less than the first neutral inner dimension ID-1 when in the neutral configuration N, which facilitates advantageous interaction with the inner tube bodies 8a, b and with the bone screw 2, as described in more detail below.

Referring now to FIG. 3D, the distal tube portion 78 is shown in the second expanded configuration E2, which can occur when the screw head 42 resides in the distal tube portion 78. As shown by comparison to FIG. 3C, the flexible portion 66 of the outer tube 6 provides significant radial expandability for increasing each of the first and second inner dimensions ID-1″, ID-2″ and the first and second outer dimensions OD-1″, OD-2″. It should be appreciated that, as used herein as a suffix for a dimension reference character, a prime (′) indicator denotes the dimension when in in the first expanded configuration E1, a double-prime (″) indicator denotes the dimension when in in the second expanded configuration E2, and a triple-prime (′″) indicator denotes the dimension when in in the third expanded configuration E3.

As mentioned above, the flexible distal portion 66 of the outer tube 6 can also flex radially inward from the neutral configuration N, which can occur when the distal tube portion 78 seats within a fixation hole. The flexible distal portion 66 can flex radially inward until circumferentially adjacent spring members 68 contact one another (i.e., until the spring relief slots 70 become circumferentially closed at the distal end 52) (not shown). It should be appreciated that the outer tube 6 is preferably configured such that when the flexible distal portion 66 is fully radially contracted, the inner dimension ID-2 of the distal tube portion 78 is sufficient to allow passage of the screw shaft 44 therethrough.

Referring now to FIGS. 4A-4B, an exemplary inner tube body 8a is shown, particularly an inner guide sleeve 8a for guiding instrumentation to the treatment site for insertion of a bone fixation member 2 within a fixation hole 7 of the bone plate 4. The inner guide sleeve 8a extends along the longitudinal direction L from a proximal end 80 to a distal end 82. The inner guide sleeve 8a also has an outer surface 88 and a radially opposed inner surface 90 that defines a cannulation 92 extending longitudinally from the proximal end 80 to the distal end 82. The inner guide sleeve 8a has an insertion portion 86 configured to extend within the cannulation 58 of the outer tube 6. In particular, the outer surface 88 of the insertion portion 86 is configured to interface with the inner surface 56 of the outer tube 6 within the main cannulation 58 thereof. The insertion portion 86 of the inner guide sleeve 8a has a proximal sleeve region 86a and a distal sleeve region 86b longitudinally spaced from each other. The distal sleeve region 86b can extend to the distal end 82 and is preferably configured to facilitate engagement with the inner surface of the fixation hole 7, as described in more detail below. As shown in FIG. 4A, the distal sleeve region 86b can have a reduced radial dimension relative to the that of the proximal sleeve region 86a. In such embodiments, the insertion portion 86 can also include an intermediate step-down region 86c between the proximal and distal sleeve regions 86a, b.

The inner guide sleeve 8a is configured to attach and lock with the outer tube 6. Accordingly, the outer tube 6 and the inner guide sleeve 8a can collectively be referred to as a “sleeve assembly.” The sleeve assembly includes a locking mechanism for locking the inner guide sleeve 8a with the outer tube 6, as discussed in more detail below. When locked together, the outer tube 6 and inner guide sleeve 8a collectively form a locked guide sleeve assembly 95, as shown in FIG. 4B. To facilitate such locking attachment, the inner guide sleeve 8a has a proximal coupling formation 84 configured to engage the coupling structure 65 of the outer tube 6, as described in more detail below. Additionally, the outer tube 6 and the inner guide sleeve 8a are cooperatively configured such that, when locked together, the distal end 82 of the inner guide sleeve 8a extends distally from the distal end 52 of the outer tube 6 at an offset distance L1, as also described in more detail below.

Referring now to FIGS. 4C-4D, the cannulation 92 of the inner guide sleeve 8a forms a secondary, inner cannulation 92 of the locked guide sleeve assembly 95 for guiding the instrumentation to the treatment site. For example, the secondary cannulation 92 can be configured to interchangeably receive one or more trocars 10, drill bits 12, measuring devices (e.g., calibrated drill bits 14), and other such instruments. As shown in FIG. 4C, when the locked guide sleeve assembly 95 receives a trocar 10 within the secondary cannulation 92, the locked guide sleeve assembly 95 can be employed to provide an incision through soft tissue 3 to the target location of the treatment site, such as to the fixation hole 7 of the bone plate 4. In this assembled configuration (with trocar 10), the locked guide sleeve assembly 95 can cut the incision until the distal end 82 of the inner guide sleeve 8a seats within the fixation hole 7. The surgeon can hold the locked guide sleeve assembly 95 at this seated position while removing the trocar 10. Subsequently, as shown in FIG. 4D, a hole opening device, such as a drill bit 12, can be inserted through the secondary cannulation 92 to extend through the fixation hole 7 and pre-drill a hole in the underlying bone 5.

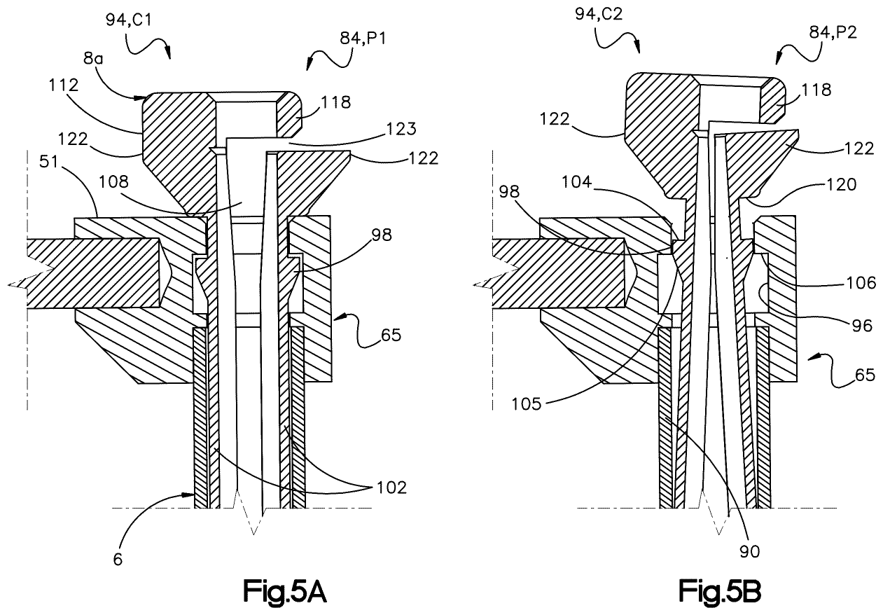

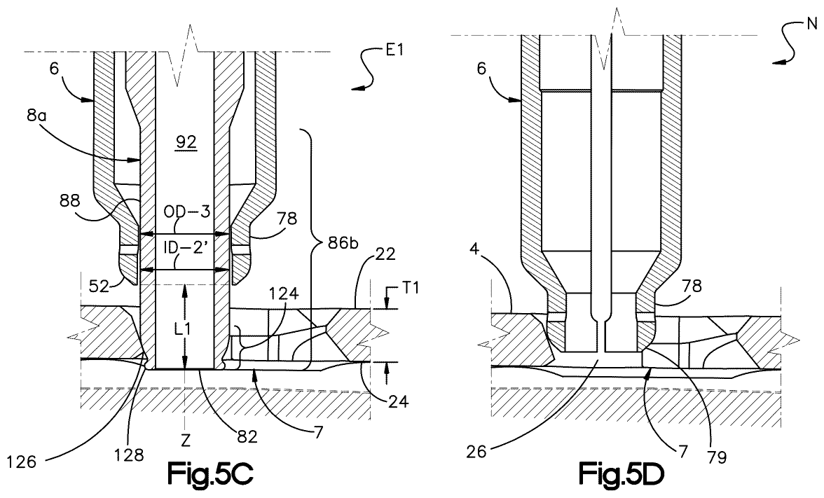

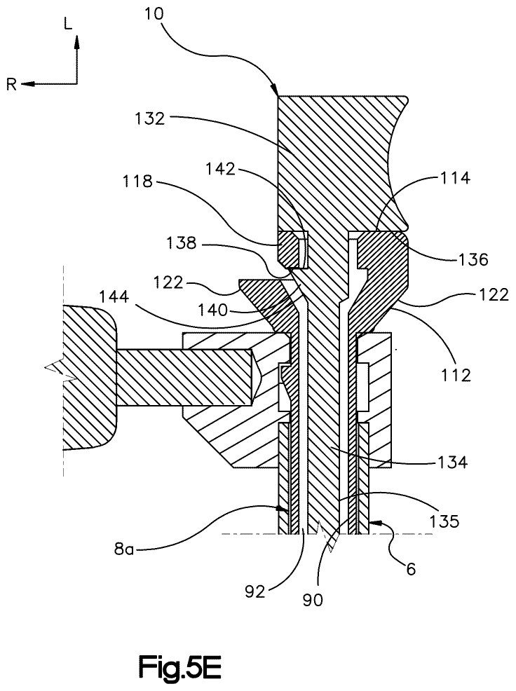

Referring now to FIGS. 5A-5B, the proximal coupling formation 84 of the inner guide sleeve 8a and the coupling structure 65 of the outer tube 6 can collectively form the locking mechanism for locking the inner guide sleeve 8a with the outer tube 6. In the illustrated embodiment, the locking mechanism is a detent mechanism 94 and is defined by the coupling structure 65 and the coupling formation 84. Thus, in such embodiments, the coupling structure 65 and the coupling formation 84 are constituent components of the detent mechanism 94. The detent mechanism 94 is configured to transition between: (1) a locked configuration C1 (shown in FIG. 5A), in which the insertion portion 86 of the inner guide sleeve 8a is longitudinally affixed within (and with respect to) the cannulation 58 of the outer tube 6; and (2) an unlocked configuration C2 (shown in FIG. 5B), in which the insertion portion 86 of the inner guide sleeve 8a is longitudinally translatable within (and with respect to) the cannulation 58 of the outer tube 6.

The detent mechanism 94 includes at least one recess 96 and at least one protrusion 98 that is configured to reside within the at least one recess 96 in the locked configuration C1 (FIG. 5A) and to be longitudinally movable into and out of the at least one recess 96 in the unlocked configuration C2 (FIG. 5B). As shown in FIG. 5A, in the locked configuration C1, the at least one protrusion 98 mechanically interferes with the associated body material that defines the at least one recess 96 so as to prevent proximal movement of the inner guide sleeve 8a relative to the outer tube 6. As shown in FIG. 5B, in the unlocked configuration C2, the at least one protrusion 98 does not mechanically interfere with the associated body material that defines the at least one recess 96, thereby allowing the inner guide sleeve 8a to move proximally relative to the outer tube 6.

To facilitate transitioning the at least one protrusion 98 and/or the at least one recess 96 between the locked and unlocked configurations C1, C2, the detent mechanism 94 includes at least one spring member 102, which is configured to flex between: (1) a first position P1, at which the detent mechanism 94 is in the locked configuration C1 (shown in FIG. 5A), and (2) a second position P2, at which the detent mechanism 94 can be in the unlocked configuration C2 (shown in FIG. 5B). Preferably, the at least one spring member 102 is configured such that the first position P1 is a neutral, unbiased position of the at least one spring member 102.

In the illustrated embodiment, the at least one protrusion 98 comprises a pair of radially opposed protrusions 98 defined by the inner guide sleeve 8a, and the at least one recess 96 is an annular recess 96 defined by the outer tube 6. The protrusions 98 define upper surfaces 104 that are configured to interface with a stop surface 106 of the outer tube 6 within the recess 96. In particular, as shown in FIG. 5A, when in the locked configuration C1 (and first position P1), the upper surfaces 104 of the protrusions 98 underlay, and thus mechanically interfere with, the stop surface 106. As shown in FIG. 5B, when in the unlocked configuration C2 (and second position P2), the upper surfaces 104 of the protrusions 98 are spaced radially inward from the stop surface 106, and are thus unimpeded from longitudinal translation by the stop surface 106.

The at least one spring member 102 of the illustrated detent mechanism 94 is at least partially defined by longitudinal spring relief slots 108 extending proximally from respective distal slot ends 110 toward the proximal end 80 of the inner guide sleeve 8a. The presence of the spring relief slots 108 provides the coupling formation 84 with a plurality of spring members or arms 102 that are circumferentially spaced from each other. As shown, the spring relief slots 108 can include a pair of radially opposed slots 108 extending through the inner guide sleeve 8a, thereby providing a first spring member 102 and an opposed second spring member 102 that can flex radially toward and away from each other. It should be appreciated that the first and second spring members 102 are respective portions of the inner guide sleeve 8a are located on opposite sides of the pair of spring relief slots 102. In additional embodiments, the inner guide sleeve 8a can include one (1), three (3), four (4), or more than four (4) spring slots 108, some of which and up to all which can be unevenly spaced from each other about the central axis of the inner guide sleeve 8a.

The protrusions 98 of the inner guide sleeve 8a also preferably include tapered lower surfaces 105 that facilitate a snap-or click-like transition to the locked configuration C1. For example, the lower surfaces 105 of the protrusions 98 taper radially inwardly and distally so as to engage the coupling structure 65, such as the proximal mounting surface 51 and/or the lead-in surface 55, as the insertion portion 86 inserts distally within the cannulation 58 of the outer tube 6. This engagement as the inner guide sleeve 8a moves distally forces the spring members 102 toward each other, thereby loading a bias force. When the protrusions 98 distally pass the stop surface 106, a return force opposite the bias force causes the spring members 102 to snap back to the first position P1. During this snap back, the spring members 102 impinge against the outer tube 6, thereby creating an audible “click” sound, which provides an audible indication (i.e., audible feedback) that the inner guide sleeve 8a is locked to the outer tube 6. It should be appreciated that the snap back can also provide a tactile indication (i.e., tactile feedback) that the inner guide sleeve 8a is locked to the outer tube 6.

The coupling formation 84 of the inner guide sleeve 8a also preferably includes a mounting head 112 at the proximal end 80 to facilitate manual manipulation of the inner guide sleeve 8a, particularly for coupling the inner guide sleeve 8a with the outer tube 6. As shown, the mounting head 112 can extend radially outward from the insertion portion 86. The mounting head 112 defines a proximal surface 114 at the proximal end 80. The proximal surface 114 can define an upper perimeter 116 of the cannulation 92. Preferably, the upper perimeter 116 extends an entire revolution about the central axis X2 in contiguous fashion with the proximal surface 114. The provide lateral space for the upper perimeter 116 of the cannulation 92, the head 112 can have an overhang portion 118 at the proximal end 80, which overhang portion 118 is positioned above the spring relief slots 108.

The mounting head 112 can include a distal mounting surface 120 configured to abut the proximal mounting surface 51 of the outer tube 6 at least when in the locked configuration C1. In this manner, engagement between the distal mounting surface 120 and the proximal mounting surface 51 prevents distal translation of the inner guide sleeve 8a when in the locked configuration. Thus, the detent mechanism 94 longitudinally affixes the inner guide sleeve 8a with the outer tube 6 when in the locked configuration C1. The mounting head 112 is also configured to facilitate manual transition of the detent mechanism 94 between the locked and unlocked configurations C1, C2. The mounting head 112 can define a pair of opposed tabs 122 on opposite sides of the spring relief slots 102. The tabs 122 are configured to be pressed radially toward each other to transition the first and second spring members 102 from the first position P1 to the second position P2 for unlocking the detent mechanism 94. The mounting head 112 can also define a lateral opening 123 that is open with, and extends radially outward from, the secondary cannulation 92, and is longitudinally positioned between an underside of the overhang portion and an associated one of the tabs 122.

It should be appreciated that, although the spring members 102 of the illustrated embodiment are defined by the coupling formation 84 of the inner guide sleeve 8a, in other embodiments the outer tube 6 can have at least one spring member to transition between the locked and unlocked configurations C1, C2. Additionally or alternatively, the one or more protrusions 98 of the detent mechanism 94 can be defined by the outer tube 6 while the one or more recesses 96 can be defined by the inner guide sleeve 8a. It should be appreciated that various adaptations can be made to the detent mechanism 94 without departing from the scope of the present disclosure.

Referring now to FIG. 5C, as mentioned above, the outer tube 6 and the inner guide sleeve 8a are cooperatively configured so that when they are locked together the distal end 82 of the inner guide sleeve 8a is distally offset at the offset distance L1 from the distal end 52 of the outer tube 6. The offset distance is preferably greater than a thickness T1 of the plate body 20 adjacent the respective fixation hole 7, as measured between the outer plate surface 22 and the bone-facing plate surface 24 along a direction parallel with the central axis Z of the respective fixation hole 7. This allows the distal end 52 of the outer tube 6 to be spaced a distance above the outer plate surface 22 when the distal end 82 of the inner guide sleeve 8a is fully seated within the fixation hole 7.

Additionally, the inner guide sleeve 8a has a distal tip region 124 configured to facilitate seating within the fixation hole 7 in a manner advantageous for opening a hole in the underlying bone 5. In particular, the distal tip region 124 can include a tapered surface 126 that tapers radially inwardly and distally and is configured to seat within the fixation hole 7, particularly in a lower axial region of the hole 7. The tapered surface 126 preferably has a rounded profile in an axial reference plane (i.e., a reference plane coextensive with the central axis X2). The tapered surface 126 provides multiple advantages for engagement with the fixation hole 7. One advantage is that the tapered surface 126 is configured to provide a centering mechanism for seating the distal end 82 centrally within the hole 7. When targeting a combi-hole, as shown, the tapered surface 126 facilitates centering within either the locking hole portion 30 or the compression hole portion 32.

The tapered surface 126 is also advantageously configured to facilitate angulation of the inner guide sleeve 8a while in contact with the interior hole surface 26. This allows the surgeon to employ the locked guide sleeve assembly 95 to target the fixation hole 7 through soft tissue, and after the surgeon receives indication (e.g., tactile indication) that the distal tip region 124 is seated within the hole 7, the surgeon can then angulate the sleeve assembly to a desired angulation with respect to the hole 7. The distal tip region 124 can also include a radial lip 128 that is distally spaced from the tapered surface 126 and is preferably contiguous with the distal end 82 of the inner guide sleeve 8a. The lip 128 is configured to limit the range of angulation with the fixation hole 7, particularly by abutting the bone-facing surface 24 of the bone plate 4 or a lower relief surface within the fixation hole 7. Moreover, the lip 128 in combination with the tapered surface 126 has also been observed to provide advantageous tactile feedback when the maximum angulation range is reached.

The distal region 86b of the inner guide sleeve 8a can define an outer dimension OD-3 that is greater than the second neutral inner dimension ID-2 of the outer tube 6. Accordingly, when the inner guide sleeve 8a is locked with the outer tube 6, the distal sleeve region 86b engages and radially expands the spring members 68 of the outer tube 6. Such radial expansion facilitates insertion of a trocar 10 and subsequently a hole opener (e.g., a drill bit 12) through the secondary cannulation 92 during use. Thus, the trocar 10 can remain fully deployed within the secondary cannulation 92 while the inner guide sleeve 8a remains fully seated within the fixation hole 7, including at a nominal orientation or at an angulated orientation. Similarly, the drill bit 12 can be deployed through the secondary cannulation 92 while the inner guide sleeve 8a remains fully seated within the fixation hole 7 (see FIG. 4D), including at a nominal or angulated orientation. In this manner, the distal sleeve region 86b protects the interior surface of the fixation hole 7 from contact with the trocar 10 and the drill bit 12.

It should be appreciated that in other embodiments, the distal tip region 124 of the inner guide sleeve 8a can have exterior threads that are configured to threadedly engage interior threads within a fixation hole 7, including at a nominal orientation or an angulated orientation. It should also be appreciated that the distal tips region 124 can have various other modifications while remaining within the scope of the present disclosure.

Referring now to FIGS. 5C-5D, another advantage provided by the interfacing geometries of the inner guide sleeve 8a and the outer tube 6 involves drop-down seating of the outer tube 6 in the fixation hole 7 after removal of the inner guide sleeve 8a. During use, with the locked guide sleeve assembly 95 extending through soft tissue to the fixation hole 7, after the drill bit 12 is deployed and removed from the secondary cannulation 92, the inner guide sleeve 8a can be unlocked and withdrawn proximally from the outer tube 6. The inventors have found that as the insertion portion 86 of the inner guide sleeve 8a withdraws proximally, particularly after the distal end 82 of the inner guide sleeve 8a proximally passes the distal tube portion 78 of the outer tube 6, the inner guide sleeve 8a no longer imparts sufficient frictional force to the outer tube 6 to maintain a longitudinal offset between the distal end 52 of the outer tube 6 and the fixation hole 7. In turn, the outer tube body 6 has sufficient mass to move distally under force of gravity through the soft tissue until the distal end 52 contacts the interior hole surface 26. Moreover, in practice, the surgeon typically applies a measure of distal force on the outer tube 6 (such as via the handle member 60) as the inner guide sleeve 8a is proximally withdrawn, which further facilitates the outer tube 6 advancing into engagement with the fixation hole 7 after the inner guide sleeve 8a withdraws proximally past the distal tube portion 78. The inventors have observed during testing that this drop-down seating of the outer tube 6 within the fixation hole 7 is virtually automatic upon withdrawal of the inner guide sleeve 8a, particularly when the outer tube 6 is within the preferred angulation range when the inner guide sleeve 8a is withdrawn. Moreover, even when the bone plate 4 is positioned horizontally or underneath the underlying bone 5 in 3D space, the drop-down seating of the outer tube 6 within the fixation hole 7 is virtually automatic when the surgeon applies a measure of distal force to the outer tube 6 during proximal withdrawal of the inner guide sleeve 8a. In this manner, the surgical system 100 described herein can advantageously provide the surgeon with a high degree of confidence that, once the distal end 82 of the inner guide sleeve 8a seats within the fixation hole 7, the outer tube 6 will, in-turn, seat centrally within the fixation hole 7.

Referring now to FIG. 5E, the trocar 10 is preferably configured to selectively lock with and unlock from the locked guide sleeve assembly 95. For this reason, the trocar 10 and the guide sleeve 8a can collectively define a secondary, trocar locking mechanism 130. The trocar 10 has a proximal head portion 132 and an insertion portion 134 that extends distally from the proximal head portion 132 and is configured to extend through the secondary cannulation 92. The insertion portion 134 of the trocar 10 defines an outer surface 135 configured to interface with the inner surface 90 of the inner guide sleeve 8a within the secondary cannulation 92. The proximal head portion 132 is configured to seat against the mounting head 112 of the inner guide sleeve 8a. In particular, the proximal head portion 132 has a distal surface 136 that can be configured to seat against the proximal surface 114 of the inner guide sleeve 8a.

The trocar locking mechanism 130 includes at least one stop surface 138 and at least one protrusion 140 that is configured to be positioned distally of, and in mechanical interference with, the at least one stop surface 138 when in a locked position. In this manner, the at least one protrusion 140 restricts proximal movement of the trocar 10 relative to the inner guide sleeve 8a when locked. In the illustrated embodiment, the at least one protrusion 140 is defined by the trocar 10 and the at least one stop surface 138 is defined by the inner guide sleeve 8a. As shown, the at least one protrusion 140 can be a single protrusion 140 extending radially outward from the outer surface 135 of the insertion portion 134 of the trocar 10. The at least one stop surface 138 can be defined on an underside of the overhang portion 118 of the mounting head 112 of the inner guide sleeve 8a. Thus, when the trocar 10 is locked with the inner guide sleeve 8a, the protrusion 140 of the trocar 10 can reside in the lateral opening 123 of the mounting head 112 between the overhang portion 118 and the associated tab 122. In such embodiments, the underside of the overhang portion 118 defines the stop surface 138.

The protrusion 140 has an upper surface 142 and a lower surface 144 longitudinally spaced from each other. The upper surface 142 of the protrusion 140 is configured to interface with the stop surface 138 when locked. The lower surface 144 of the protrusion 140 tapers radially inwardly so as to engage structure of the mounting head 112, such as the upper perimeter 116 of the secondary cannulation 92 and/or a lead-in surface into the secondary cannulation 92, as the insertion portion 134 of the trocar 10 inserts distally within the secondary cannulation 92. This engagement forces the spring members 102 of the inner guide sleeve 8a that defines the overhang portion 118 toward the other spring member 10, thereby loading a bias force. When the protrusion 140 distally passes the stop surface 138, a return force opposite the bias force causes the spring member 102 to snap back to a neutral position, thereby creating an audible “click” sound, which provides audible feedback (and also tactile feedback) that the trocar 10 is locked to the inner guide sleeve 8a. To unlock the trocar 10 from the inner guide sleeve 8a, the tab 122 associated with the overhang portion 118 can be pressed radially toward the opposite tab 122, thereby moving the stop surface 138 so as to be radially outwardly spaced from the protrusion 140, thereby allowing the trocar 10 to be withdrawn distally from the inner guide sleeve 8a. It should be appreciated that the foregoing trocar locking mechanism 130 is an exemplary example of a locking mechanism for locking the trocar 10 with the inner guide sleeve 8a, and that various alternate designs for locking the trocar 10 to the inner guide sleeve 8a are within the scope of the present disclosure.

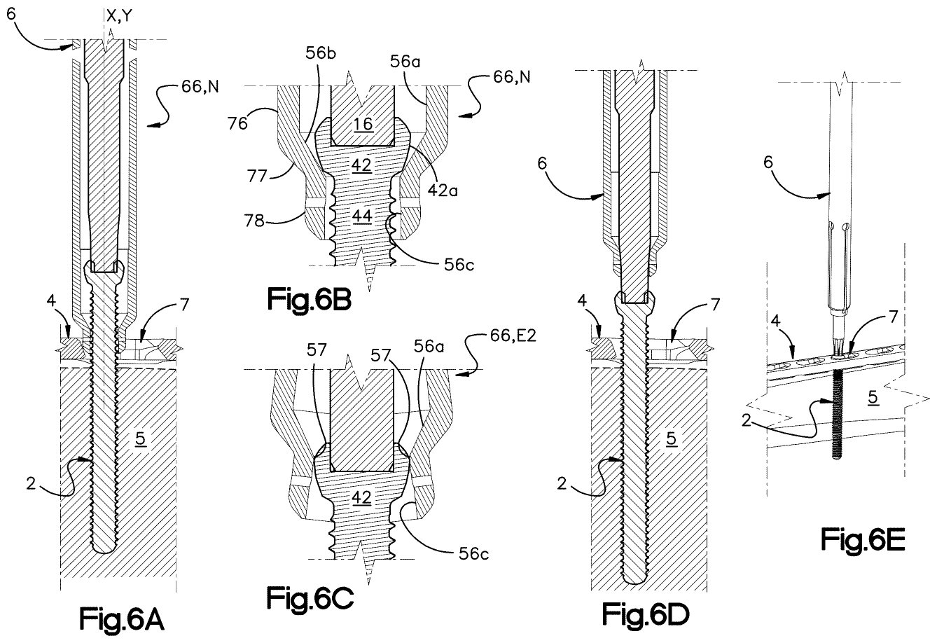

Referring now to FIGS. 6A-6E, bone screw 2 insertion through the outer tube 6 will now be described. With the outer tube 6 seated within the fixation hole 7 and the inner guide sleeve 8a removed (and preferably with the bone hole pre-drilled or otherwise pre-formed or opened), the outer tube 6 can be employed for inserting the bone screw 2 through the fixation hole 7 and into the underlying bone 5. Referring now to FIG. 6A, the outer tube 6 is configured for use with bone screws 2 having a maximum head outer dimension D2 (FIG. 2C) that is no greater than, and preferably only marginally less than, the first neutral inner dimension ID-1 of the main tube portion 76 (FIG. 3C). Thus, the bone screw 2 can be inserted into the proximal opening 53 of the cannulation 58 and, as shown in FIG. 6A, advanced distally along the cannulation 58 by a driver 16.

Referring now to FIGS. 6A-6B, the screw 2 is preferably sized with the outer tube 6 so that the maximum shaft outer dimension D1 (e.g., major thread diameter) (FIG. 2B) is substantially equivalent to, or only marginally less than, the second neutral inner dimension ID-2 of the main tube portion 76 (FIG. 3C). In this manner, sliding engagement between the outer surface of the screw shaft 44 and the inner surface 56 of the main tube portion 76 and can function as a centering mechanism for the bone screw 2, particularly by maintaining the screw axis Y in substantially coaxial alignment with the central axis X of the outer tube 6. It should also be appreciated that the tapered inner surface 56b of the step-down tube portion 77 is configured to center the screw shaft 44 along the central axis X should the screw 2 become misaligned during insertion through the main tube portion 76.

As shown in FIG. 6B, the tapered inner surface 56b of the step-down tube portion 77 is configured to contact the distal head surface portion 42b as the screw head 42 distally approaches the distal tube portion 78. This contact can provide a further centering mechanism to align the screw 2 along the central axis X. Additionally, the interfacing geometries between the tapered inner surface 56b and the distal head surface portion 42b are configured to initiate and provide a smooth radial expansion of the flexible tube portion 66 as the screw head 42 advances distally in contact with the tapered inner surface 56b during screw insertion. In particular, the interfacing geometries of the tapered inner surface 56b and the distal head surface portion 42b are configured to reduce a longitudinal component of a normal force between the screw head 42 and the step-down tube portion 77 as the screw head 42 traverses the step-down tube portion 77. As shown in FIG. 6C, in the illustrated embodiment, maximum radial expansion of the flexible tube portion 66 occurs when the location of the maximum outer dimension D2 of the screw head 42 (FIG. 2C) contacts an edge boundary 57 between the inner surfaces 56b, c of the step-down tube portion 77 and the distal tube portion 78. Further distal screw 2 advancement from this position causes the flexible tube portion 66 to contract radially back toward the neutral configuration N.

It should be appreciated that the smooth radial expansion, in combination with the geometry of the tapered distal tip surface 79, allows the flexible tube portion 66 to expand even when fully seated within a fixation hole 7, including a fixation hole 7 having locking structures (e.g., threads) therein, such as within a locking hole portion 30 of a combi-hole. In particular, during such expansion, the tapered distal tip surface 79 is configured to ride upward and outwardly along the locking structures (e.g., internal threads) of the fixation hole 7.

Referring now to FIG. 6D, after the screw shaft 44 advances through the fixation hole 7 and engages underlying bone material 5 to a sufficient depth, the design of the flexible tube portion 66 provides the surgeon with the option of proximally retracting the outer tube 6 before the screw head 42 is seated within the fixation hole 7. In such optional uses, the screw 2 can be further distally driven by the driver 16 from the position shown in FIG. 6D until the screw head 42 is fully seated within the fixation hole 7, as shown in FIG. 6E. Thus, it should be appreciated that the outer tube 6 described herein provides the surgeon with flexible options for driving the bone screw 2 to affix the bone plate 4 to underlying bone 5.

Referring now to FIG. 7A-7B, an exemplary inner tube body 8b in the form of an inner removal sleeve 8b is shown. As described above, the inner removal sleeve 8b is configured to facilitate accessing and removing a bone fixation member 2 (e.g., bone screw 2) that was previously implanted at the treatment site. The removal sleeve 8b extends along the longitudinal direction L from a proximal end 150 to a distal end 152. The removal sleeve 8b also has an outer surface 154 and a radially opposed inner surface 156 that defines a cannulation 158 extending longitudinally from the proximal end 150 to the distal end 152. The removal sleeve 8b has a proximal mounting formation 160 configured to mount with the outer tube 6, and also includes an insertion portion 162 configured to extend within the cannulation 58 of the outer tube 6. In this manner, the outer tube 6 and the removal sleeve 8b collectively form constituent parts of a removal sleeve assembly 97. Similar to the inner guide sleeve 8a described above, the outer surface 154 of the insertion portion 162 of the removal sleeve 8b is configured to interface with the inner surface 56 of the outer tube 6 within the main cannulation 58 thereof, such that the cannulation 158 of the removal sleeve 8b forms an inner, secondary cannulation 158 of the removal sleeve assembly 97. The secondary cannulation 158 is configured for passage of one or more removal instruments therein, such as a driver 16, to advance distally to the treatment site and engage the bone screw 2, and subsequently to retrieve and remove the bone screw 2 proximally. The removal sleeve 8b can optionally be configured so that the distal end 152 thereof extends distally from the distal end 52 of the outer tube 6 at an offset distance L2 when the inner removal sleeve 8b is fully seated with the outer tube 6. In other embodiments, the distal end 152 of the removal sleeve 8b can be substantially flush with, or slightly proximally spaced from, the distal end 52 of the outer tube 6 when the removal sleeve 8b is coupled with the outer tube 6.

Referring now to FIG. 7C, the proximal mounting formation 160 of the inner removal sleeve 8b has a distal mounting surface 164 that can be configured to seat against the outer tube 6, such as against the proximal mounting surface 51 thereof. In the illustrated embodiment, the removal sleeve 8b need not lock with the outer tube 6, although in other embodiments the removal sleeve 8b can be configured to selectively lock with and unlock from the outer tube 6.

Referring now to FIG. 7D, the distal end 152 of the inner removal sleeve 8b can be configured to seat against the bone plate 4, such as against the outer plate surface 22 thereof, or partially or entirely against the interior surface 26 within the fixation hole 7. Although the distal end 152 of the removal sleeve 8b is shown seated against the bone plate 4 at a nominal orientation, it should be appreciated that the distal end 152 can seat against the bone plate 4 (including partially or entirely against the interior surface 26 of the hole 7) at an angulation A1 within the angulation range described above. It should be appreciated that during a screw removal procedure, before coupling with the removal sleeve 8b, the outer tube 6 can be employed as part of a guide sleeve assembly 95 (i.e., locked with an inner guide sleeve 8a) with a trocar 10 also locked thereto to create an incision through the soft tissue 3 to the target bone screw 2. The surgeon can retain the outer tube 6 in place in the soft tissue while unlocking and removing the trocar 10 and the inner guide sleeve 8a from the main cannulation 58, and can subsequently insert and advance the removal sleeve 8b distally through the cannulation 58 to the treatment site. Alternatively during such a screw removal procedure (and before coupling with the removal sleeve 8b), a driver 16 (e.g., screw driver) can be inserted within the cannulation 58 instead of a trocar 10, such that a distal tip of the driver 16 extends distally from the distal end 52 and can be employed to create an incision through the soft tissue.

The insertion portion 162 of the removal sleeve 8b defines an outer dimension OD-4, measured radially at the outer surface 154, and defines an inner dimension ID-4, measured radially at the inner surface 156. The inner dimension ID-4 is at least equivalent to, and preferably greater than, the maximum head outer dimension D2. The outer surface 154 of the removal sleeve 8b imparts a bias force against the inner surface 56c of the distal portion 78 of the outer tube 6, thereby radially expanding the flexible portion 66 of the outer tube 6 to a third expanded configuration E3. In the third expanded configuration, the distal portion 78 of the outer tube 6 defines an expanded second inner dimension ID-2′″, measured radially between opposing portions of the inner surface 56c. In the illustrated embodiments herein, the expanded second inner dimension ID-2′″ in the third configuration E3 is greater than those of the first expanded configuration E1 (ID-2′, caused by contact with the inner guide sleeve 8a, see FIG. 5C) and the second expanded configuration E2 (ID-2″, caused by contact with the outer surface 48 of the screw head 42 at the maximum head outer dimension D2, see FIGS. 3D and 6C). In this manner, the flexibility of the outer tube 6 allows the removal sleeve 8b to have a wider inner dimension ID-4 for easy removal of the bone screws 2 of various size.

With reference to FIGS. 8A-8D, use of the removal sleeve assembly 97 for removing a bone screw 2 from a treatment site will now be described. As shown in FIG. 8A, with the removal assembly 97 fully inserted and the distal end 152 of the removal sleeve 8b engaged with the bone plate 4 and surrounding the screw head 42, a driver 16 can be advanced distally through the secondary cannulation 158 of the removal sleeve 8b until the distal end of the driver 16 engages the drive socket 46 of the screw head 42. As shown in FIG. 8B, the driver 16 can counter-rotate in a manner backing the screw shaft 44 from the underlying bone 5 and thereby proximally retracting the screw head 42 within the secondary cannulation 158. As shown in FIG. 8C, after the screw head 42 proximally passes the distal portion 78 of the outer tube 6, the removal sleeve 8b can be withdrawn from the cannulation 58 of the outer tube 6. This, in-turn, causes the flexible portion 66 of the outer tube 6 to flex radially inwardly back to the neutral configuration N. The surgeon can continue to counter-rotate the driver 16 until the screw shaft 44 disengages from the underlying bone 5 and preferably also proximally passes the fixation hole 7.

Referring now to FIG. 8D, after the screw shaft 44 disengages from the underlying bone 5, and with the screw head 42 located proximal of the distal tube portion 78, the driver 16 can be withdrawn from the cannulation of the outer tube 6. When the screw head 42 is positioned within the intermediate step-down tube portion 77, the inner surface 56b thereof effectively cradles the distal head surface portion 42b of the screw head 42. The flexible tube portion 66 preferably has a spring force in the radial direction such that, when the screw head is positioned in the intermediate step-down tube portion 77 and the screw shaft 44 extends through the distal tube portion 78 (see also FIG. 6B), the outer tube 6 retains the screw head 42 therein against the force of gravity. Stated differently, the flexible tube portion 66 has sufficient spring force to prevent the screw 2 from dropping out of the outer tube 6, at least when the screw head 42 is located proximal of the distal tube portion 78. Preferably, the flexible tube portion 66 has sufficient radial spring force to hold the screw head 42 within the cannulation while the outer tube 6 is proximally withdrawn through the soft tissue 3 and the screw is entirely removed therefrom.

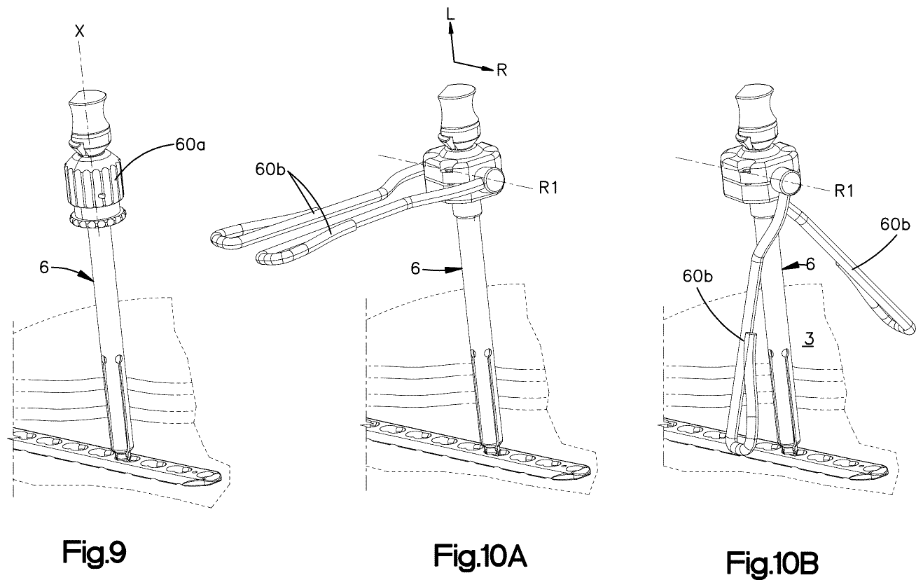

It should be appreciated that various modifications can be made to the outer tube 6 and the inner tube bodies 8a, b described above without departing from the scope of the present disclosure. For example, some modified handle features of the outer tube body 6 are described below with reference to FIG. 9 and FIGS. 10A-10B.

Referring now to FIG. 9, the outer tube body 6 can have a knob-like handle member 60a, which can extend an entire revolution about the central axis X. Thus knob-like handle member 60a can be particularly well suited for providing a small, manipulatable handle with finger-grip features arranged around an outer grip surface in coaxial fashion with the central axis.

Referring now to FIG. 10, the outer tube body 6 can have a pair of rotatable handle arms 60b that can be individually rotated about a radial axis R1 oriented along the radial direction R. The rotatable handle arms 60b can be configured to rotate about the radial axis R1, such as for being placed into contact with an outer skin surface of the patient. The rotatable handle arms 60b can also be configured to selectively lock in place, such as for providing a self-supporting mechanism for the outer tube 6 with respect to patient anatomy.

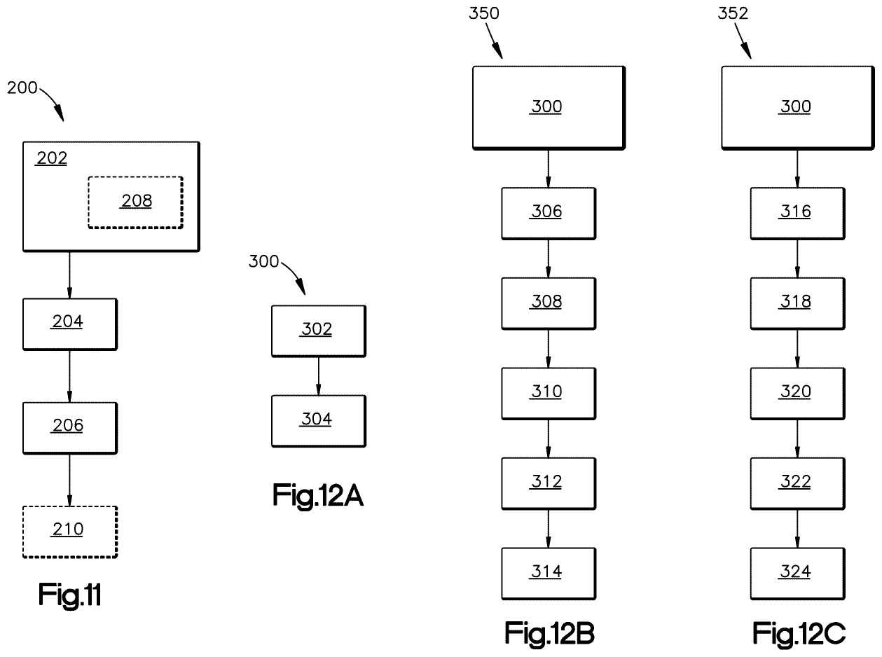

With reference to FIGS. 11-12C, exemplary methods relating to the surgical system 100 will be described.

Referring now to FIG. 11, an exemplary method 200 for preparing a sleeve assembly 95, 97 for use will now be described. The method 200 includes steps 202, 204, and 206. Step 202 includes inserting an inner tube body 8a, b within a cannulation 58 of an outer tube body 6 along a central axis X of the outer tube body 6 such that an insertion portion 86 of the inner tube body 8a, b engages an expandable distal portion 66 of the outer tube body 6 and a distal end 82, 152 of the inner tube body 8a, b extends distally from a distal end 52 of the outer tube body 8 at an offset distance L1. Step 202 can be performed ex vivo, in vivo, or a combination thereof.

Step 204 includes expanding the expandable distal portion 66 along a radial direction R perpendicular to the central axis X via the engagement with the insertion portion 86 of the inner tube body 8a. After step 204 is complete, a bone fixation member 2 is translatable along the central axis X and through the cannulation 58. Step 206 includes orienting the outer and inner tube bodies 6, 8 relative to soft tissue such that the central axis X intersects a fixation hole 7 of a bone plate 4 and underlying bone 5. Step 204 and 206 can each be performed ex vivo, in vivo, or a combination thereof.

It should be appreciated that step 202 can include an optional sub-step 208 of abutting a distal mounting surface 120, 164 of a proximal mounting formation 112, 160 of the inner tube body 8a, b against a proximal mounting surface 51 of the outer tube body. This abutting sub-step 208 can conclude step 202.

In exemplary method 200, the expandable distal portion 66 of the outer tube body 6 can comprise a plurality of spring arms 68 that are defined circumferentially between a plurality of spring relief slots 70 that are defined by the outer tube body 6 and extend longitudinally to the distal end 52 of the outer tube body 52, as described above with reference to FIGS. 3A-3B. In such instances, the step 204 of expanding and the expandable distal portion 66 of the outer tube body comprises engaging inner surfaces 56 of the spring arms 68 with an outer surface 88, 154 of a distal portion of the inner tube body 8a, b, thereby flexing the spring arms 68 radially outwardly.

Method 200 can be performed using the inner guide sleeve 8a as the inner tube body. In such uses, method 200 can include a step 210 of advancing an instrument 10 distally along a second cannulation 92 of the inner tube body 8a, which can be performed before or after step 206, and can be performed Step 202 ex vivo, in vivo, or a combination thereof. Method 200 can also include a step 212 of locking a proximal head portion of the instrument 10 with a proximal mounting formation 112 of the inner tube body 8a while the instrument 10 extends distally along the second cannulation 92.

Referring now to FIG. 12A, an exemplary method 300 for providing passage for a bone fixation member through soft tissue will now be described. The method 300 includes steps 302, 304, and 306. Step 302 includes inserting an inner tube body 8a, b within a cannulation 58 of an outer tube body 6 along a central axis X of the outer tube body 6 such that an insertion portion 86 of the inner tube body 8a, b engages and expands an expandable distal portion 66 of the outer tube body 6 along a radial direction R perpendicular to the central axis X. Step 302 can be performed ex vivo, in vivo, or a combination thereof. At the conclusion of step 302, a distal end 82, 152 of the inner tube body 8a, b extends distally from a distal end 52 of the outer tube body 8 at an offset distance L1. Additionally, after step 304, a bone fixation member 2 is translatable along the central axis X and through the cannulation 58. Step 304 includes advancing the outer and inner tube bodies 6, 8a, b in unison along a trajectory along the central axis X through soft tissue 3 toward an associated fixation hole 7 of a bone plate 4 that interfaces with underlying bone 5 until the distal end 82, 152 of the inner tube body 8a, b contacts the bone plate 4 without or adjacent to the fixation hole 7.

Referring now to FIG. 12B, one exemplary use 350 of the foregoing method 300 is for inserting a bone fixation member 2 within patient anatomy. In this exemplary use, the inner tube body 8a, b is an inner guide sleeve 8a. Accordingly, during step 304 of this exemplary use 350, the distal end 82 of the inner guide sleeve 8a contacts the bone plate 4. Additionally, this exemplary use 350 includes steps 306, 308, and 310. Step 306 includes advancing a drill bit 12 distally through a cannulation 92 of the inner guide sleeve 8a and through the fixation hole 7 and into the underling bone 5, thereby pre-drilling a hole in the underlying bone 5. Step 308 includes withdrawing the drill bit 12 proximally from the cannulation 92 of the inner guide sleeve 8a. Step 310 includes withdrawing the inner guide sleeve 8a proximally at least partially along the cannulation 58 of the outer tube body 6, thereby allowing the outer tube body 6 to advance distally into contact with the bone plate 4 within the fixation hole 7.

Exemplary use 350 can also include steps 312 and 314. Step 312 includes translating a bone fixation member 2 distally through the cannulation 58 of the outer tube body 6 toward the fixation hole 7. Step 314 includes driving the bone fixation member 2 distally so that a shaft 44 of the bone fixation member 2 engages bone material along an interior surface of the pre-drilled hole in the underlying bone 5.

Referring now to FIG. 12C, another exemplary use 352 of the foregoing method 200 is for removing a bone fixation member 2 from patient anatomy. In this additional exemplary use 352, the inner tube body 8a, b is a removal sleeve 8b. Exemplary use 352 includes steps 316 and 318. Step 316 includes inserting a driver 16 distally through a cannulation 158 of the inner tube body 8b such that a distal end of the driver 16 engages a drive socket 46 defined in a head 42 of a bone fixation member 2 that is seated within the fixation hole 7. Step 318 includes counter-rotating the driver 16, thereby backing the bone fixation member 2 proximally from the underlying bone 5 such that the head 42 advances proximally into the cannulation 158 of the removal sleeve 8b.

This additional exemplary use 352 can also include steps 320, 322, and 324. Step 320 includes translating the head 42 of the bone fixation member 2 proximally along the cannulation 158 of the removal sleeve 8b so that the head 42 passes a distal tube portion 78 of the outer tube body 6 and enters an intermediate step-down tube portion 77 of the outer tube body 6 that is distally spaced from a main tube portion 76 of the outer tube body 6 (see FIG. 6B). After step 320 is complete, step 322 is performed, which includes removing the removal sleeve 8b proximally from the cannulation 58 of the outer tube body 6, thereby causing the expandable distal portion 66 to flex radially inwardly such that an inner surface 56b of the intermediate step-down tube portion 77 cradles a distal surface portion 42b of the head 42. Step 324 includes withdrawing the outer tube body 6 proximally from the soft tissue 3. During step 324, the intermediate step-down tube portion 77 retains the head 42 therein such that the bone fixation member 2 is removed from the soft tissue 3.

It should be appreciated that the foregoing methods 200 and uses 301, 302 can include additional and/or alternative steps while remaining within the scope of the present disclosure. It should also be appreciated that the sequence of various steps in the foregoing methods 200, 300 can be adjusted as needed.

It should be appreciated that the various features of the surgical system 100 described above (such as the outer tube 6, inner guide sleeve 8a, removal sleeve 8b, and bone screw 2, by way of non-limiting examples) are provided as exemplary features for adapting inserting and/or removing a bone fixation member into or from patient anatomy. These parameters can be adjusted as needed without departing from the scope of the present disclosure.

It should also be appreciated that in additional embodiments, the surgical system 100 and various components thereof can be provided in a kit that includes a plurality of interchangeable components (e.g., outer tubes 6, inner guide sleeves 8a, removal sleeves 8b, bone fixation members 2, bone plates 4, trocars 10, drill bits 12, measuring instruments (e.g., calibrated drill bits 14), and drivers 16) having different sizes and surgical indications, such that the surgeon can select the particular components for treating the patient.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A surgical system, comprising:
an outer tube body elongate along a longitudinal direction, the outer tube body having a proximal end and a distal end spaced from the proximal end in a distal direction oriented along the longitudinal direction, the outer tube body defining a cannulation extending from the proximal end to the distal end along a central axis oriented along the longitudinal direction, the outer tube body having a flexible portion that extends to the distal end and defines at least one spring member configured to allow the flexible portion to flex in a radial direction perpendicular to the central axis between:
a neutral configuration, in which the distal end of the outer tube body defines a first radial dimension, and
a expanded configuration, in which the distal end of the outer tube body defines a second radial dimension greater than the first radial dimension; and
an inner tube body having an insertion portion configured to insert within the cannulation of the outer tube body and contact the flexible portion of the outer tube body in a manner that flexes the flexible portion into the expanded configuration.

2. The surgical system of claim 1, wherein the at least one spring member comprises a plurality of spring arms that extend to the distal end of the outer tube body, and the spring arms are defined by a plurality of longitudinal spring relief slots defined by the outer tube body and extending distally to the distal end of the outer tube body.

3. The surgical system of claim 1, wherein:
the outer tube body has an outer surface and an inner surface opposite each other in the radial direction, the inner surface defines the cannulation, the outer tube body comprising:
a main tube portion defining a first inner dimension along the radial direction; and
a distal tube portion that is distally spaced from the main tube portion and extends to the distal end, the distal tube portion defining a second inner dimension along the radial direction, wherein the second inner dimension is less than the first inner dimension when in the neutral configuration, and
the insertion portion of the inner tube body defines an outer dimension along the radial direction that is less than the first inner dimension and greater than the second inner dimension, such that the insertion portion engages the inner surface of the outer tube body along the distal tube portion to force the flexible portion from the neutral configuration to the expanded configuration.

4. The surgical system of claim 3, further comprising a bone plate having a plate body defining an outer plate surface configured to face away from an underlying bone and a bone-facing surface configured to face the underlying bone, the plate body defining one or more holes that extend from the outer plate surface to the bone-facing surface along one or more respective central hole axes, wherein the one or more holes are configured for receiving one or more respective bone fixation members for affixing the bone plate to the underlying bone,
wherein the distal end of the inner tube body is configured to seat within a respective hole of the one or more holes such that the distal end of the outer tube body is proximally spaced from the respective hole substantially at an offset distance when in the locked configuration, wherein the offset distance is greater than a thickness of the plate body adjacent the respective hole, wherein the thickness is measured between the outer plate surface and the bone-facing plate surface along a direction parallel with a central axis of the fixation hole.

5. The surgical system of claim 4, wherein:
the respective hole extends through the plate body along a central hole axis, the respective hole defines an inner hole dimension along a second radial direction perpendicular to the central hole axis, and the inner hole dimension is measured at an intermediate axial location along central hole axis between the outer plate surface and the bone-facing surface, and
the distal portion of the outer tube body defines an outer dimension along the radial direction, wherein the outer dimension is substantially equivalent to the inner hole dimension when in the neutral configuration such that the distal end of the outer tube body is configured to seat within the respective hole when in the neutral configuration.

6. The surgical system of claim 3, further comprising:
a bone fixation member having a head and a shaft extending distally from the head; and
a driver configured to advance the bone fixation member distally through the cannulation of the outer tube body and to drive the shaft into bone material located distally from the distal of the outer tube body,
wherein the driver and the bone fixation member are insertable through the outer tube member while the inner tube member is removed from the outer tube member,
wherein the shaft defines an outer shaft dimension along the radial direction that is less than the first and second inner dimensions, and
wherein the head defines an outer head dimension that is less than the first inner dimension and greater than the second inner dimension of the outer tube body, such that the head engages the inner surface along the distal portion of the outer tube body to force the flexible portion from the neutral configuration to the expanded configuration as the head advances distally through the cannulation.

7. The surgical system of claim 6, wherein the at least one spring member is configured such that, when the shaft resides in the distal tube portion and the head resides proximal of the distal tube portion of the outer tube body, the outer tube body retains the head therein against the force of gravity.

8. The surgical system of claim 3, wherein the inner tube body is a first inner tube body, and the system further comprises a second inner tube body that is insertable within the cannulation of the outer tube body in interchangeable fashion with the first inner tube body, wherein the second inner tube body has a distal portion that defines an outer radial dimension that is less than the first inner dimension and greater than the second inner dimension of the outer tube body, such that the distal portion of the second inner tube body is configured to engage the inner surface along the distal portion of the outer tube body to force the flexible portion from the neutral configuration to the expanded configuration, wherein the second inner tube body defines a second cannulation configured for passage of a bone fixation member longitudinally through the second cannulation.

9. The surgical system of claim 1, wherein:
the outer tube body defines a coupling structure, and the inner tube body defines a coupling formation configured to engage the coupling structure of the outer tube body; and
the coupling structure and the coupling formation collectively define a detent mechanism that is configured to transition between:
an unlocked configuration, in which the insertion portion of the inner tube body is longitudinally translatable within the cannulation of the outer tube body, and
a locked configuration, in which the insertion portion is longitudinally affixed within the cannulation, such that a distal end of the inner tube body is spaced distally at an offset distance from a distal end of the outer tube body.

10. A method for preparing a sleeve assembly to provide passage for a fixation member, the method comprising:
inserting an inner tube body within a cannulation of an outer tube body along a central axis of the outer tube body such that an insertion portion of the inner tube body engages an expandable distal portion of the outer tube body and a distal end of the inner tube body extends distally from a distal end of the outer tube body at an offset distance;
expanding the expandable distal portion of the outer tube body along a radial direction perpendicular to the central axis via the engagement with the insertion portion of the inner tube body, wherein a fixation member is translatable along the central axis and through the cannulation after the expanding step; and
orienting the outer tube body, wherein the central axis intersects a fixation hole of a plate after the inserting step.

11. The method of claim 10, wherein the inserting step comprises abutting a distal mounting surface of a proximal mounting formation of the inner tube body against a proximal mounting surface of the outer tube body, wherein the abutting step concludes the inserting step.

12. The method of claim 10, wherein:
the expandable distal portion of the outer tube body comprises a plurality of spring arms that are defined circumferentially between a plurality of spring relief slots, the spring relief slots are defined by the outer tube body and extend longitudinally to the distal end of the outer tube body, and
the expanding step comprises engaging inner surfaces of the spring arms with an outer surface of a distal portion of the inner tube body, thereby flexing the spring arms radially outwardly.

13. The method of claim 10, comprising:
before or after the orienting step, advancing an instrument distally along a second cannulation of the inner tube body; and
locking a proximal head portion of the instrument with a proximal mounting formation of the inner tube body while the instrument extends distally along the second cannulation.

14. The method of claim 13, further comprising:
unlocking the proximal head portion of the instrument from the proximal mounting formation of the inner tube body;
removing the instrument proximally from the second cannulation; and
advancing a second instrument through the second cannulation and through the fixation hole.

15. The method of claim 10, wherein the inner tube body defines a second cannulation defining an inner dimension along a radial direction perpendicular to the central axis, wherein the inner dimension of the second cannulation is greater than a maximum outer radial dimension of a head of the bone fixation member.

16. The method of claim 10, wherein the outer tube body comprises a main tube portion, a distal tube portion that extends longitudinally to the distal end of the outer tube, and an intermediate step-down tube portion that extends longitudinally between the main tube portion of the distal tube portion.

17. The method of claim 16, further comprising:
positioning the fixation member such that 1) a head of the fixation member resides within or is located distally of the intermediate step-down tube portion, and 2) a shaft of the bone fixation member extends through the distal tube portion; and
removing the inner tube body from the cannulation of the outer tube body, thereby causing the expandable distal portion to flex radially inwardly,
wherein, after the removing step:
an inner surface of the intermediate step-down tube portion cradles a distal surface portion of the head, and
the outer tube body retains the head therein against the force of gravity.

18. The method of claim 16, further comprising:
contacting a distal end of the inner tube body against the plate, such that the distal end of the outer tube body is proximally spaced from the plate substantially at the offset distance; and
withdrawing the inner tube body proximally along the cannulation of the outer tube body such that the distal end of the inner tube body passes the distal tube portion, thereby causing:
the expandable distal portion to flex radially inwardly; and
the outer tube body to advance distally into contact with in interior surface of the plate within the fixation hole.

19. The method of claim 18, further comprising:
translating a driver and a bone fixation member attached to the end of the driver distally through the cannulation toward the fixation hole; and
driving the bone fixation member distally so that 1) a shaft of the bone fixation member extends through the fixation hole, and 2) a head of the fixation member seats within the fixation hole.

20. The method of claim 19, wherein the translating step comprises causing the head to engage the inner surface of the intermediate step-down tube portion in a manner flexing the distal flexible portion radially outward.
